SEQUENCE LISTING (25 sequences)

SEQ ID NO: 1 - RNA, 49 nt, Artificial
  Dual hairpin-terminal RNA targeted to the F9 target sequence of MMP-9.
aagcgcaagcuuggugagaggugccggaugcugucccucgagcaaucga

SEQ ID NO: 2 - DNA, 49 bp, Artificial
  Dual hairpin-terminal DNA targeted to the F9 target sequence of MMP-9.
aagcgcaagcttggtgagaggtgccggatgctgtccctcgagcaatcga

SEQ ID NO: 3 - RNA, 14 nt, Artificial
  Antisense RNA targeted to the F9 target site of MMP-9.
gaggugccggaugc

SEQ ID NO: 4 - DNA, 40 bp, Homo sapiens
cgacgtgaatggcatccggcacctctatggtcctcgccct

SEQ ID NO: 5 - DNA, 40 bp, Homo sapiens
gctgcacttaccgtaggccgtggagataccaggagcggga

SEQ ID NO: 6 - RNA, 14 nt, Artificial
  Antisense RNA targeted to the F9 target region of MMP-9 with 2′ O-methyl.
gaggugccggaugc

SEQ ID NO: 7 - DNA, 14 bp, Artificial
  Dual hairpin-terminal DNA sequence targeted to the F9 target sequence of MMP-9 having a phosphodiester backbone.
gaggtgccggatgc

SEQ ID NO: 8 - RNA, 19 nt, Artificial
  5′ Strand of siRNA targeted to the F9 target sequence of MMP-9.
uggcauccggcaccucuau

SEQ ID NO: 9 - RNA, 19 nt, Artificial
  3′ Strand of siRNA targeted to the F9 target sequence of MMP-9.
accguaggccguggagaua

SEQ ID NO: 10 - RNA, 14 nt, Artificial
  F9 RNA invert.
cguaggccguggag

SEQ ID NO: 11 - DNA, 62 bp, Artificial
  Multiple cloning site for use in making deletion libraries for use in design of the targeting sequence of the oligonucleotides of the invention.
gcttggtgatgcattcgatatcgtttaaacgcccgggcgcggccgcggcgcctccagtcgac

SEQ ID NO: 12 - DNA, 24 bp, Artificial
  Portion of a multiple cloning site for use in making deletion libraries for use in design of the targeting sequence of the oligonucleotides of the invention.
gtcgacgggactgcaggtttaaac

SEQ ID NO: 13 - DNA, 23 bp, Artificial
  Portion of a multiple cloning site for use in making deletion libraries for use in design of the targeting sequence of the oligonucleotides of the invention.
gaagacagtcaccaagcttcagc

SEQ ID NO: 14 - DNA, 5658 bp, Artificial
  pBK
ctagttctggcgcagaaccatggcctttgtccagtttaactggggacaaggccaagattcctaggctcgcaaaacatgtctgtcatgcactttccttcctgaggtcatggtttggctgcattccatgggtaagcagctcctccctgtgagtcatgcactttccttcctgaggtcatggtttggctgcattcccctgtgagtcatgcactttccttcctgaggtcatggtttggctgcattccatgggtaagcagctcctccctgtggcctttttttttataatatataagaggccgaggccgcctctgcctccaccctttctctcaagtagtaagggtgtggaggctttttctgaggcctagcaaaactatttggggaaatccctattcttttgcaatttttgcaaaaatggataaagttcttaacagggaagaatccatggagctcatggaccttttaggccttgaaagagctgcctggggaaatcttcccttaatgagaaaagcttatttaaggaagtgtaaggaatttcatcctgacaaagggggcgacgaggataaaatgaagagaatgaatactttgtataaaaaaatggagcaggatgtaaaggtagctcatcagcctgattttggaacttggagtagctcagaggtttgtgctgattttcctctttgcccagataccctgtactgcaaggaatggcctatttgttccaaaaagccttctgtgcactgcccttgcatgctatgtcagcttagattaaggcatttaaatagaaaatttttaagaaaagagcccttggtttggatagattgctactgcattgactgcttcacacagtggtttggcttagacctaactgaagaaactctgcaatggtgggtccaaataattggagaaactcccttcagagatctaaagctttaaggtaactaacttatatttagataaataataaaatattaaaaggccctaagtaattattttttttataggtgccaacctatggaacagaagagtgggagtcctggtggagttcctttaatgaaaaatgggatgaagatttattttgccatgaagatatgtttgccagtgatgaagaagcaacagcagattctcaacactcaacaccacccaaaaaaaaaagaaaggtagaagaccctaaagactttccctctgatctacaccagtttcttagtcaagctgtatttagtaatagaacccttgcctgctttgctgtgtatactactaaagaaaaagctcaaattctgtataaaaaacttatggaaaaatattctgtaacttttattagtagacacatgtgtgctgggcataatattatattctttttaactccacatagacatagagtttctgcaattaataatttctgtcaaaagctgtgtacctttagttttttaatttgtaagggtgttaataaggaatacttactatatagtgccttaactagagatccataccatactatagaagaaagcattcaagggggcttaaaggagcatgattttagcccagaagagcctgaagaaacaaagcaggtgtcttggaaattaattactgagtatgcagtagagacaaagtgtgaggatgtgtttttattattaggtatgtatttagaatttcaatacaatgtagaggagtgtaaaaagtgtcagaaaaaagaccagccttatcactttaagtatcatgaaaagcactttgcaaatgctattatttttgcagaaagtaaaaatcaaaaaagtatttgtcagcaagcagtagatacagttttagctaaaaaaagagtagatacccttcatatgaccagggaagaaatgctaacagaaagattcaatcatatattagataaaatggatttaatatttggagctcatggaaatgctgtactagaacaatatatggcaggtgttgcttggctgcactgtttgctacctaaaatggattctgtaatatttgattttttgcactgtattgttttcaatgtacctaaaagaagatactggttatttaaaggtcccattgatagtggaaaaacgacactagctgccgggttattagatttgtgtggtggtaaagccttaaatgtaaacctacccatggaaaggctaacctttgagctaggtgtagctatagatcagtacatggttgtttttgaagatgtaaaagggacaggagctgaatcaaaggatttgccttcaggacatggaataaacaatttagacagtttgagagattatttagatggaagtgttaaggtaaatttagaaaagaaacatttaaacaaaagaacccaaatatttccaccaggcttggttacaatgaatgagtatcctgtccctaaaaccctgcaagctagatttgtaagacaaatagattttaggcccaaaatatatttaagaaaatccttacaaaactcagagttcttacttgaaaaaagaattttacaaagtggaatgaccttgttgctactgctaatttggtttaggcctgtagctgattttgcaactgatatacaatctagaattgttgaatggaaggaaaggctggattctgagataagtatgtatactttttcaaggatgaaatataatatatgcttggggaaatgtattcttgatattacaagagaagaggattcagaaactgaagactctggacatggatcaagcactgaatcccaatcacaatgctcttcccaagtctcagatacttcagcccctgctgaagattcccaaaggtcagacccccatagtcaagagttgcatttgtgtaaaggctttcagtgttttaaaaggcctaaaacaccacccccaaaataacacaagcttaaaagtggcttatacaaaagcagcatttattaaatgtatatgtacaataaaagcacctgtttaaagcattttggtttgcaattgtccctgtttgtcaatatatcttatcatatctgggtcccctggaagtaactagatgatccgctgtggaatgtgtgtcagttagggtgtggaaagtccccaggctccccagcaggcagaagtatgcaaagcatctcaattagtcagcaaccaggtgtggaaagtccccaggctccccagcaggcagaagtatgcaaagtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggcgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagctaatcatcggcatagtatatcggcatagtataatacgactcactataggagggccaccatggccaagttgaccagtgccgttccggtgcttaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgaccgacgccgaccaacaccgccggggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgacggcggcccacgggtcgaattgcgcttccctgatgagaccgaaaggtcgaaagtcgaaagactcggaagcgaaagcttggtgatgcattcgatatcgtttaaacgcccgggcgcggccgcggcgcctccagtcgacgaaagtcggtctgccgaaaggcactgatgagtccgaaaggacgaaaccgacttgctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatcatgcggcccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagc

SEQ ID NO: 15 - DNA, 178 bp, Artificial
  An oligonucleotide with sequences capable of forming stable terminal hairpin loops flanked by two cis-acting ribozymes.
gagctcgcttccctgatgagtccgaaaggacgaaagtcgaaagactcggaagcgaaagcttggtgatgcattcgatatcgtttaaacgcccgggcgcggccgcggcgcctccagtcgacgaaagtcggtctgccgaaaggcactgatgagtccgaaaggacgaaaccgacttggtacc

SEQ ID NO: 16 - DNA, 8705 bp, Artificial
  pShuttle
tcgagcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagccgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagacccaagcttggtaccgagctcggatccactagtaacggccgccagtgtgctggaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccaggatcccccgccgccggacgaactaaacctgactacggcatctctgccccttcttcgctggtacgaggagcgcttttgttttgtattggtcacggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagggcccttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaatt

SEQ ID NO: 17 - RNA, 39 nt, Artificial
  F-9 dL (delta linker)
aagcgccaagcuugaggugccggaugcucgagcaaucga

SEQ ID NO: 18 - RNA, 30 nt, Artificial
  F-9 dL-min (delta linker, minimal hairpins)
ccucgcaagaggugccggaugcgcaagcau

SEQ ID NO: 19 - RNA, 49 nt, Artificial
  F-9 p
aagcgcaagcuuggugagaggugccggaugcugucccucgagcaaucgn

SEQ ID NO: 20 - DNA, 53 bp, Artificial
  F-9 dL (delta-linker) oligonucleotide
aagcgcaagcuuggugatagaggugccggaugccaugucccucgagcaaucga

SEQ ID NO: 21 - RNA, 32 nt, Artificial
  F-9 R3′L
gaggugccggaugcugucccucgagcaaucga

SEQ ID NO: 22 - DNA, 22 bp, Artificial
  F-9 R3 dL
gaggugccggaugcgcaatacg

SEQ ID NO: 23 - RNA, 32 nt, Artificial
  F-9 R3′LP
gaggugccggaugcugucccucgagcaaucgn

SEQ ID NO: 24 - DNA, 32 bp, Artificial
  F-9 R3′L
gcauccggcaccucuguccctcgagcaaucga

SEQ ID NO: 25 - DNA, 32 bp, Artificial
  F-9S R3′LP
gcauccggcaccucuguccctcgagcaaucgn

(19) United States
(12) Patent Application Publication
(10) Pub. No.: US 2004/0077082 A1
(43) Pub. Date: Apr. 22, 2004

(54) RNA-based inhibitory oligonucleotides

(76) Inventors: Richard K. Koehn, Salt Lake City, UT (US); Duane E. Ruffner, Salt Lake City, UT (US); Ramesh K. Prakash, Salt Lake City, UT (US)

Correspondence Address: MADSON & METCALF, GATEWAY TOWER WEST, SUITE 900, 15 WEST SOUTH TEMPLE, SALT LAKE CITY, UT, 84101

(21) Appl. No.: 10/273,678

(22) Filed: Oct. 18, 2002

(51) Int. Cl. C07H 21/02; C07H 21/04; A61K 48/00; C12N 5/02

(52) U.S. Cl. 435/375; 536/23.1; 514/44

(57) ABSTRACT

The invention discloses a class of sequence-specific oligonucleotides for use in silencing genes. Specifically, the invention includes targetable oligonucleotides composed of RNA, DNA, nucleic acid analogs, or some combination of the above which have a configuration such that their introduction to a solution, cell, tissue, or organism containing the target gene causes silencing of the gene to which they are targeted. The invention also includes methods of silencing a gene by exposing a solution, cell, tissue, or organism with a compound comprising an oligonucleotide of the invention. Additionally, the invention provides recombinant vectors comprising nucleic acid molecules that code for the targeted oligonucleotides of the invention.

### RELATED APPLICATIONS

This application is related to and claims the benefit of U.S. patent application Ser. No. 09/647,344 of Zhidong Chen, Duane E. Ruffner, and Michael L. Pierce filed Dec. 4, 2000 and entitled “Directed Antisense Libraries,” which is incorporated herein by reference.

### BACKGROUND OF THE INVENTION

1. Field of the Invention

The present invention relates to oligonucleotides used in gene silencing technologies. More specifically, the present invention relates to oligonucleotides with 5′- and 3′-terminal hairpins that may be configured to regulate the expression of a gene.

2. Description of Related Art

Diseases such as some cancers and many hereditary diseases have often been linked to the problems stemming from the presence or absence of crucial proteins. In some situations, the problem is the production or non-production of a protein. In others, possible problems include the production of a defective protein as a result of a mutation in the nucleic acid code for the protein, or the over- or under-production of a protein due to problems with cellular systems regulating the production or destruction of the protein. As these causes of disease have come to light, researchers have sought out compounds and methods for modulating the expression of specific genes for use in the development of novel medications.

Researchers have also sought gene silencing compounds and methods for use in deciphering the function of the thousands of proteins encoded in the DNA of many organisms. In such research uses, gene-silencing technologies may be used to “knock out,” or block the expression of specific genes by a variety of mechanisms. The phenotype resulting from lack of the gene product may then be studied in order to help discern the function of the protein encoded by the targeted nucleic acid. Such gene silencing technologies could additionally provide novel methods of treating diseases characterized by the expression of a flawed protein or the misexpression of a normal protein.

One of the first such technologies developed was the generation of mutant organisms in which a mutation had been produced which caused the affected gene to produce a non-functional or “null” allele. In these approaches, genes are knocked out by generating mutations in the genome of the selected organism using a variety of methods. Those organisms containing a mutation in the desired gene may then be selected for using known screening methods. Some such “knockout” organisms (often mice) may have mutations which may be stably passed to subsequent generations of the organism. Though knockout methods have been used successfully for years, these methods are very often expensive to practice and may require years for the generation of a single successful knockout organism.

More recently, researchers have explored methods of using a molecule capable of binding a nucleic acid encoding a specific protein and preventing its transcription or translation. Generally the molecule is a short length of RNA, DNA, or chimeric RNA/DNA commonly referred to as an antisense oligonucleotide. These oligonucleotides are complementary to a segment of the nucleic acid. In use, these oligonucleotides are administered to a cell or tissue desired to be treated, and are taken into the cell or tissue. Following this, the oligonucleotides associate with and bind to a region of the nucleic acid such as an mRNA encoding the protein to which they are complementary, thus impeding translation. This binding prevents normal translation of the nucleic acid by a number of different mechanisms, including preventing proper interaction with cellular machinery such as DNA transcription enzymes or RNA translation enzymes, and even, in some cases, targeting the nucleic acid for destruction.

Antisense technology is regarded by many as a powerful technology since antisense oligonucleotides may be targeted to a specific nucleic acid, and even to a selected region on that nucleic acid. This prevents interference with the transcription or translation of non-targeted genes. The sequence to which an antisense oligonucleotide is targeted is generally referred to as a target sequence. Because antisense oligonucleotides may be so carefully targeted to these target sequences, antisense oligonucleotides may be used to provide compositions such as medications that have near-absolute specificity, high efficacy, low toxicity, and few side effects.

In many antisense applications, however, it has been difficult to locate effective target sequences on a specific gene. Part of this difficulty stems from the fact that although there are generally a large number of potential antisense oligonucleotides available for any gene selected for targeting, only a few of these turn out to be effective. This may be due, at least in part, to the final folded structure of the nucleic acid, which may block access to regions of the sequence, rendering oligonucleotides targeted to those regions ineffective. As a result, to date, the effectiveness of proposed antisense oligonucleotides has generally been determined by lengthy and labor-intensive trial-and-error methods. These methods must be repeated for each gene desired to be targeted.

In addition to the speed and effectiveness problems encountered in antisense, many difficulties have been encountered in effectively administering antisense oligonucleotides. Further, antisense-mediated down-regulation or silencing of a gene is generally not heritable, and is in some cases effective for only a short time period in an organism.

In recent years, RNA interference (“RNAi”) technologies have become increasingly commonplace in the laboratory as a less-expensive, and in some cases more effective, alternative to prior gene-silencing technologies. RNAi is currently thought to be a process that harnesses a widely conserved biological response to cellular exposure to exogenous dsRNA to drive selective destruction of a targeted mRNA in a cell, thus effectively silencing a gene.

The mechanisms for RNAi have only recently begun to be partially understood. In RNAi, a cell is exposed to a double-stranded RNA (or “dsRNA”) sequence complementary to or identical to a target sequence on a cellular RNA such as an mRNA. See, e.g., Paddison et al., Genes & Dev., 16:948-958 (2002). This dsRNA, often referred to as a silencing trigger, is recognized by an RNAse III family nuclease called Dicer. Id. As suggested by its name, Dicer cleaves the trigger dsRNA into short 21-23-nucleotide pieces called small interfering RNAs (“siRNAs”). Id. These siRNAs are then recognized by the RNA-induced silencing complex (“RISC”), which then locates substrates based on their similarity to the siRNA. Id. Those substrate nucleic acids identified are subsequently destroyed.

Researchers have also shown that in some organisms, gene silencing may be accomplished by introducing siRNAs directly to a cell, tissue, or organism. Hannon et al., Nature, 418: 244-251 (2002). Such use is likely to have therapeutic potential since, as with antisense technology, it entails the introduction of a relatively small molecule to silence a gene.

Since they are nucleotide-based, however, siRNAs themselves may not find direct application as small molecule medicines. Such nucleotide-based compounds are often unstable in vivo. This potentially diminishes the efficacy of such agents. Their size may also cause difficulty in assuring proper administration of the compound, as well as extra costs in synthesis.

Accordingly, a need exists for novel molecular effectors of gene silencing such as oligonucleotides for silencing a gene. It would therefore be an improvement in the art to provide oligonucleotides for use in gene silencing that are sequence-specific, easily administered, and highly effective in silencing a targeted gene. It would be a further improvement in the art to provide methods of using such oligonucleotides.

Such oligonucleotides and methods for their use are disclosed herein.

### SUMMARY OF THE INVENTION

The present invention has been developed in response to the present state of the art, and in particular, in response to the problems and needs in the art that have not yet been fully solved by currently available components and methods for silencing specific genes. Thus, the present invention provides novel compounds and methods for their use in silencing a selected gene.

The invention thus provides a class of oligonucleotides that may be configured to target a specific gene for silencing. Specifically, the invention includes oligonucleotides composed of RNA, DNA, nucleic acid analogs, or some combination of the above which have a configuration such that their introduction to a cell, tissue, or organism causes silencing of the gene to which they are targeted.

The oligonucleotides of the invention include at least two primary components. Specifically, the oligonucleotides include a targeting region, and a hairpin loop. In some embodiments of the invention, the targeting region has either a 3′ hairpin loop, or a 5′ hairpin loop. In other embodiments of the invention, the targeting region has both a 3′ hairpin loop and a 5′ hairpin loop. The hairpin loops of the oligonucleotides of the invention may either be contiguous with the targeting sequence, or they may instead be coupled to the targeting sequence by intervening linker sequences. In some configurations, the sequences of the targeting region and the hairpin loop region or regions of the oligonucleotide may overlap to minimize the size of the oligonucleotide. The oligonucleotides may be varied in size and composition as discussed below to effect sequence-specific gene silencing.

The targeting sequence of the oligonucleotides of the invention is generally a length of nucleic acid between about 8 and about 50 nucleotides in length. Although the oligonucleotides of the invention may include a targeting sequence equal in size to the entire sequence of the target nucleic acid, in some more preferred embodiments of the oligonucleotides of the invention, the targeting sequence is between about 10 and about 20 nucleotides in length. Still more preferably, the targeting sequence is between about 14 and about 18 nucleotides in length.

The targeting sequence is selected to cause the silencing of a specific gene. In order to accomplish this, the targeting sequence is either substantially identical to or substantially complementary to the sequence of a target region on the gene. This target region may be selected by a variety of methods, including those described in International Patent Application No.: PCT/US99/06742, which teaches methods for locating effective antisense target regions on a gene desired to be targeted.

The targeting sequence is linked on its 3′ and 5′ ends of nucleotides to sequences that enable the formation of hairpin structures. Generally these sequences include sets of inverted complementary sequences positioned relatively near to each other on the oligonucleotide. These inverted complementary sequences pair to form a hairpin loop structure. Those portions of the oligonucleotide that pair form a double-stranded region termed the “stem” of the hairpin loop. In the oligonucleotides of the invention, the number of paired nucleotides, and thus the length of this “stem” region may be varied in length from about 1 set of paired nucleotides to about 12 sets of paired nucleotides. More preferably, however, the stem region comprises from about 2 to about 10 sets of paired nucleotides, and still more preferably from about 4 to about 6 sets of paired nucleotides in length. In some embodiments of the invention, the targeting sequence may overlap, and thus function as a part of, a portion of the stem region of one, either, or both of the hairpin structures of the oligonucleotides.

In addition to the stem-forming portions of the hairpin loop, the hairpin loop sequence includes a loop sequence. This loop sequence is a set of nucleotides positioned between the inverted complementary sequences of the hairpin stem. The loop sequence does not fold and pair like those in the stem portion of the oligonucleotide. Instead, this portion of the oligonucleotide bulges out from the stem to form a loop-shaped structure upon binding of the repeats of the stem. In some situations, it may be desirable to vary the loop sequence in size to produce loop structures of sizes varying from about 1 nucleotide to about 10 nucleotides. More preferably, it is desirable to produce a loop structure of from about 2 to about 8 nucleotides. Still more preferably, the loop structure may be from about 4 to about 6 nucleotides.

In other embodiments of the invention, the single hairpin loop, or either or both of the hairpin loops in the dual-hairpin oligonucleotides may be coupled to the targeting sequence of the oligonucleotides by linker sequences. In such oligonucleotides, the linker sequences of both the 3′ and 5′ hairpin loops may share a uniform length, or they may differ in size. Such linker sequences generally each have a length of from about 1 to about 10 nucleotides in length. Despite this, however, these linker sequences may vary in length from about 4 to about 8 nucleotides in length. In some specific oligonucleotides, the linker sequences are from about 5 to about 6 nucleotides in length.

When administered to a cell or tissue, the oligonucleotides of the invention silence the expression of a gene having a sequence identical or complementary to that of the targeting sequence of the oligonucleotide. Without being limited to any one theory, this silencing appears to be due to knockdown of the mRNA transcribed from the gene.

The invention further includes methods of silencing a gene in a cell including the steps of contacting the cell with a compound comprising an oligonucleotide of the invention including a targeting sequence and a hairpin loop at either or both of the 3′ and 5′ ends of the targeting sequence.

The present invention also provides recombinant vectors comprising nucleic acid molecules that code for the targeted hairpin oligonucleotides of the invention. In some embodiments of the invention, these recombinant vectors are plasmids. These recombinant vectors may be constructed as prokaryotic or eukaryotic expression vectors. The nucleic acid coding for the targeted hairpin oligonucleotides of the invention may be operably linked to a heterologous promoter. Additionally, the present invention further provides host cells comprising a nucleic acid that codes for the targeted hairpin oligonucleotides of the invention.

These and other features and advantages of the present invention will become more fully apparent from the following description and appended claims, or may be learned by the practice of the invention as set forth hereinafter.

### BRIEF DESCRIPTION OF THE DRAWINGS

In order that the manner in which the above-recited and other features and advantages of the invention are obtained will be readily understood, a more particular description of the invention briefly described above will be rendered by reference to specific embodiments thereof which are illustrated in the appended drawings. These drawings depict only typical embodiments of the invention and are not therefore to be considered to be limiting of its scope. The invention will be described and explained with additional specificity and detail through the use of the accompanying drawings, in which:

FIG. 1A shows a MCS sequence (SEQ ID NO: 11) used in methods for generating libraries of antisense oligonucleotides suitable for use in the targeting sequence of the hairpin-terminal oligonucleotides of the invention;

FIG. 1B shows a second MCS sequence (SEQ ID NO: 12, SEQ ID NO: 13) used in methods for generating libraries of antisense oligonucleotides suitable for use in the targeting sequence of the hairpin-terminal oligonucleotides of the invention;

FIG. 2A shows the pBK expression vector (SEQ ID NO: 14) designed for episomal expression in mammalian cells encoding a hairpin-terminal oligonucleotide according to the invention;

FIG. 2B shows the hairpin-terminal oligonucleotide (SEQ ID NO: 15) encoded by the pBK expression vector (SEQ ID NO: 14) of FIG. 2A with cis-acting ribozymes used to liberate the hairpin-terminal oligonucleotide from the larger transcript;

FIG. 3 shows the pShuttle expression vector (SEQ ID NO: 16) designed for episomal expression in mammalian cells encoding a hairpin-terminal oligonucleotide according to the invention

FIG. 4A shows an RNA oligonucleotide of the invention (SEQ ID NO: 1) with 5′ and 3′ terminal hairpin loops targeted to the F9 target region of MMP-9;

FIG. 4B shows a phosphorothioate DNA oligonucleotide of the invention (SEQ ID NO: 2) with 5′ and 3′ terminal hairpin loops targeted to the F9 target region of MMP-9;

FIG. 5 is a photograph of a PCR gel showing the results of an assay using the oligonucleotides of FIG. 4A (SEQ ID NO: 1) and FIG. 4B (SEQ ID NO: 2) to inhibit the expression of MMP-9 in HT1080 cells;

FIG. 6A shows an RNA oligonucleotide of the invention (SEQ ID NO: 1) with 5′ and 3′ terminal hairpin loops targeted to the F9 region of MMP-9;

FIG. 6B shows an antisense RNA oligonucleotide (SEQ ID NO: 3) targeted to the F9 region of MMP-9;

FIG. 6C shows the result of an in vitro assay of MMP-9 inhibition by the antisense oligonucleotide of FIG. 6B (SEQ ID NO: 3) compared with the inhibition brought about by the oligonucleotide with terminal hairpin loops of FIG. 6A (SEQ ID NO: 1);

FIG. 7A is an illustration of the F9 antisense target and the F9 RNAi target of MMP-9 on a segment of the MMP-9 gene sequence;

FIG. 7B shows the oligonucleotides used in an assay conducted to compare their effectiveness in silencing the MMP-9 gene;

FIG. 8 is a photograph of an ethidium bromide-stained electrophoresis gel showing the results of PCR with MMP-9- and glyceraldehyde phosphate dehydrogenase—(GAPDH) specific PCR primers showing MMP-9- and GAPDH-specific PCR fragments;

FIG. 9 is a bar graph showing a plot of the ratio of the intensities of MMP-9 to GAPDH from the gel of FIG. 8.

FIGS. 10A through 10J are exemplary structures of oligonucleotides of the invention (SEQ ID NOS: 17-25).

FIG. 11 shows the result of a matrigel invasion assay comparing the function of the F9 RNA with that of psDNA and siRNAs.

### DETAILED DESCRIPTION OF THE PREFERRED EMBODIMENTS

The practice of the present invention will employ, unless otherwise indicated, conventional methods of virology, microbiology, molecular biology, and recombinant DNA techniques within the skill of the art. Such techniques are fully explained in the literature. See, e.g., Sambrook, et al., Molecular Cloning: A Laboratory Manual (Current Edition); DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tijessen, ed.); Fundamental Virology, 2nd Edition, vol. I & II (B. N. Fields & D. M. Knipe, eds.).

All publications, patents and patent applications cited herein, whether supra or infra, are hereby incorporated by reference in their entirety. As used in this specification and the appended claims, the singular forms “a,” “and,” and “the” include plural references unless the content clearly dictates otherwise.

The present invention relates to oligomeric compounds for modulating the function of specific nucleic acid molecules encoding a selected gene product. More specifically, the invention relates to single-stranded oligomeric compounds with at least one 3′ or 5′ terminal hairpin that silence a selected gene in a sequence-specific manner. Without being limited to any one theory, it is thought that the silencing is brought about by mRNA knockdown of the mRNA encoding the gene product of the selected gene. The invention further includes compositions comprising such oligomeric compounds, including pharmaceutical compounds, and methods for their use. The invention also includes vectors encoding the oligonucleotides of the invention, as well as host cells transfected with these expression vectors. The invention additionally includes methods of silencing a gene by administering the oligomeric compounds of the invention.

In the context of this application, the term “oligonucleotides” is used to refer to an oligomer or polymer of ribonucleic acid (RNA), deoxyribonucleic acid (DNA), or analogs thereof. This term includes oligonucleotides composed of naturally-occurring nucleotides, sugars and internucleotide (or “backbone”) linkages, as well as oligonucleotides having modified nucleotides, sugars, or backbone linkages, as well as oligonucleotides having mixed natural and modified nucleotides, sugars, and backbones or other non-naturally occurring portions that have similar function to naturally-occurring compounds.

The present invention also includes recombinant vectors including nucleic acid sequences that code for the targeted hairpin oligonucleotides of the invention. These recombinant vectors may be plasmids, and may be constructed as prokaryotic and eukaryotic expression vectors. The vectors may additionally include a heterologous promoter operably linked to the nucleic acid sequence coding for the targeted hairpin oligonucleotides of the invention.

Single-stranded antisense oligonucleotides have commonly been used to block expression of genes in the art. It is understood, however, that many antisense oligonucleotides fail to function for any of a number of reasons, including inability to achieve proper binding with the target nucleic acid, and instability in the presence of cellular nucleases.

Double-stranded RNA oligonucleotides are used in RNA interference (“RNAi”) techniques to knock down the mRNA of a specifically-targeted gene. In RNAi, double-stranded RNA molecules (“dsRNAs”) are introduced into a cell. Following introduction to the cell, the dsRNA molecule is recognized by Dicer, an RNAse III-family nuclease. Dicer enzymatically cuts the dsRNA molecule into small double-stranded pieces of from about 21 to about 23 nucleotides in length. These short strands are called small interfering RNAs (“siRNAs”).

Following the step of dsRNA processing by Dicer, the resultant siRNAs are recognized by a RNA-induced silencing complex (“RISC”). RISC then identifies cellular mRNAs which are homologous to the siRNAs. These homologous mRNAs are then destroyed, resulting in a functional silencing of the targeted gene in the system.

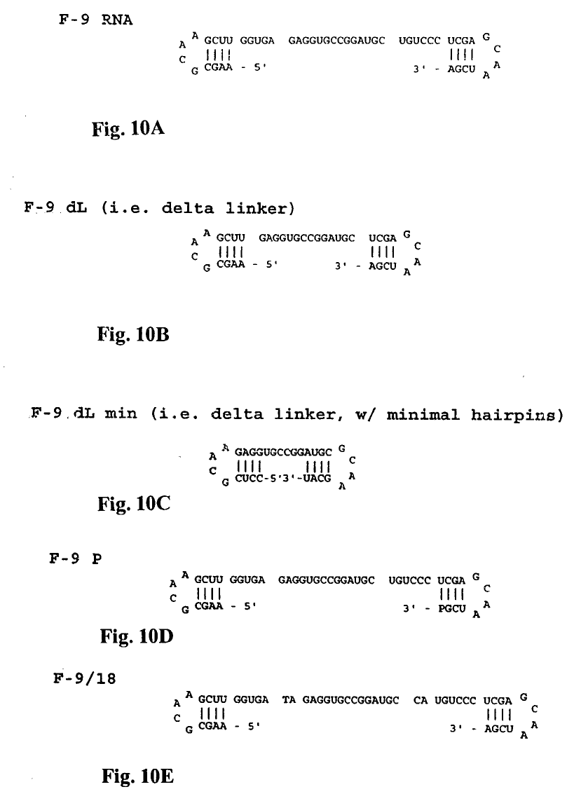

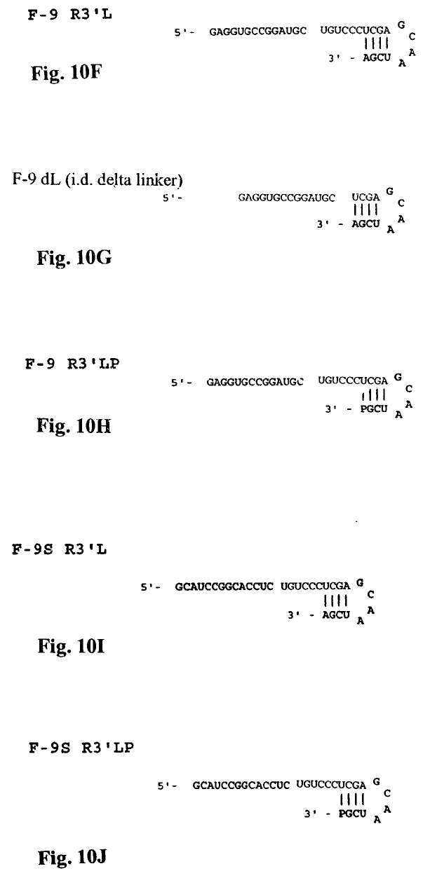

The present invention provides single-stranded oligonucleotides with at least one 3′ or 5′ terminal hairpin loop. Some oligonucleotides include a single hairpin, and other oligonucleotides of the invention include terminal hairpin loops on both the 3′ and 5′; ends. FIGS. 10A through 10J include exemplary structures of oligonucleotides of the invention. FIG. 10A shows a targeted oligonucleotide (SEQ ID NO: 1) having a 5′ hairpin which includes a 5′ loop and a 5′ stem. Similarly, the oligonucleotide includes a 3′ hairpin with a 3′ loop and a 3′ stem. The 5′ and 3′ hairpins are linked to the targeting sequence of the oligonucleotide by 5′ and 3′ linker sequences. In this embodiment, the 5′ linker sequence includes 5 nucleotides, and the 3′ linker sequence includes 6 nucleotides.

Referring now to FIG. 10B (SEQ ID NO: 17), an additional example of the oligonucleotides is shown. This oligonucleotide is the oligonucleotide of FIG. 10A with the 5′ and 3′ linker sequences omitted. FIG. 10C (SEQ ID NO: 18) shows yet another embodiment of the oligonucleotides of the invention, this time overlapping the targeting sequence with portions of the stem regions of the 5′ and 3′ hairpins. FIG. 10D (SEQ ID NO: 19) shows an oligonucleotide having a puromycin substituted at the end of the 3′ hairpin. FIG. 10E (SEQ ID NO: 20) shows an oligonucleotide having an extended targeting region of 18 nucleotides.

The oligonucleotides of the invention also include oligonucleotides having a single terminal hairpin, as shown in exemplary oligonucleotides shown in FIGS. 10F through 10J. FIG. 10F shows an oligonucleotide (SEQ ID NO: 21) having a targeting sequence with a linker attached to its 3′ end, and a 3′ hairpin having a loop and a stem attached to the linker. FIG. 10G shows an oligonucleotide (SEQ ID NO: 22) similar to that of FIG. 10F, omitting the linker sequence. FIG. 10H (SEQ ID NO: 23) shows an oligonucleotide having a puromycin substituted at the end of the single 3′ hairpin. FIGS. 10I (SEQ ID NO: 24) and 10J (SEQ ID NO: 25) show additional oligonucleotides having a 3′ terminal hairpin and an alternative sense targeting sequence. FIG. 10J shows this sequence having a 3′ terminal puromycin.

These “hairpin-terminal” oligonucleotides have been shown to knock down gene expression at a specific target more efficiently than either antisense or RNAi oligonucleotides targeted to the same target region. In addition, the oligonucleotides of the invention are sequence-specific. The oligonucleotides of the invention appear to be useful with regard to a wide variety of genes, and may be varied in composition to provide a specifically-targeted compound suitable for use in vivo and in vitro.

The oligonucleotides of the invention first include a targeting sequence targeted to a target region of a selected nucleic acid. As used herein, the term “targeted to,” is intended to include polynucleotides at least substantially identical to or complementary to at least a portion of the selected nucleic acid. DNA or cDNA encoding a specific protein is thus included, as is RNA such as pre-mRNA, mRNA (“messenger RNA”), ssRNA (“single-stranded RNA”), shRNA (“short-hairpin RNA”), siRNA (“small interfering RNA”), dsRNA (“double-stranded RNA”), and hybrid nucleic acids such as artificial sequences having at least a portion of the sequence of a specific protein. Further, an oligonucleotide may be “targeted to” a selected nucleic acid functionally, i.e., by assaying its complementarity to a target sequence and selecting oligonucleotides by their function. Such oligonucleotides targeted to a selected nucleic acid sequence may thus be obtained from a library produced using random library generation methods and screened for complementarity or identity to at least a portion of the target sequence.

In addition, the terms “nucleic acid,” “target nucleic acid,” and “nucleic acids encoding a specific protein” also include sequences having any of the known base analogs of DNA and RNA such as, but not limited to, 4-acetylcytosine, 8-hydroxy-N6-methyladenosine, aziridinylcytosine, pseudoisocytosine, 5-(carboxyhydroxylmethyl) uracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methyl guanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5oxyacetic acid methylester, 2,6-diaminopurine and 2′ modified analogs such as, but not limited to O-methyl-, amino-, and fluoro-modified analogs.

In some embodiments of the invention, the targeting sequences are selected from directed antisense libraries constructed to allow selection of effective antisense target sequences on a nucleic acid desired to be silenced. As used herein, the term “antisense oligonucleotide” denotes an oligonucleotide that is complementary to, and thus has the capacity to specifically hybridize with, a nucleic acid. This is especially used herein to refer to oligonucleotides whose binding modulates the normal activity or function of the target nucleic acid.

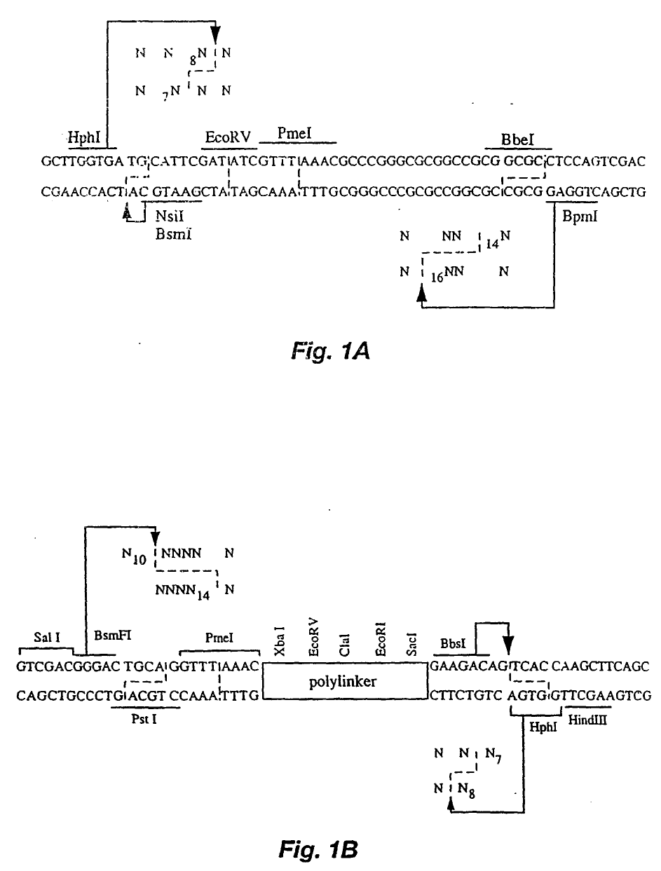

The construction of suitable directed antisense libraries for use in the selection of targeting sequences may be conducted by a procedure that requires the use of specially designed bacterial and/or mammalian plasmid vectors. Herein, the term “vector” is used to denote any genetic element, such as a plasmid, phage, transposon, cosmid, chromosome, virus, virion, or other such element known in the art which is capable of replication when associated with the proper control elements and which can transfer sequences between cells. Thus, the term includes cloning and expression vehicles, as well as viral vectors. These vectors are configured to possess a specially designed multi-cloning sequence (“MCS”). Illustrative MCSs are shown in FIGS. 1A (SEQ ID NO: 11) and 1B (SEQ ID NO: 12, SEQ ID NO: 13).

The procedure uses the multi-cloning sequence and a series of enzymatic manipulations to produce DNA fragment libraries directed against any desired gene of interest. The fragment libraries contain all possible overlapping fragments spanning the entire length of the gene of interest. In vitro or in vivo transcription of each of these DNA fragments allows the production of an antisense RNA molecule targeted to the site on the RNA transcript that is encoded by the DNA fragment. Transcription of the entire DNA fragment library produces all possible antisense RNA molecules targeting all positions on the RNA target. Expression of the library in mammalian cells allows identification of effective target sites for antisense-mediated gene inhibition.

In the procedure, the MCS is placed in a suitable circular plasmid vector, and a blunt-ended DNA fragment encoding the gene of interest is ligated into the EcoRV-digested MCS. Since the gene can be inserted in one of two orientations, a clone is selected, according to methods known in the in art such as nucleotide sequencing or restriction mapping, wherein the gene insert is suitably oriented. The orientation of the insert will be chosen such that the antisense strand of the insert will be transcribed by an adjacent promoter.

A deletion library is next prepared. One of skill in the art would recognize that many suitable methods exist for preparing a deletion library. In one exemplary method, the plasmid containing the gene of interest is digested with both PmeI and BbeI. The Bbel terminus is protected from exonuclease III digestion because of its 3′ overhang, while the PmeI terminus is a suitable substrate for digestion. The digested plasmid is then treated with exonuclease III, and aliquots are removed over time into a stop mixture. The time points are chosen such that deletions are generated after every nucleotide across the entire gene. After exonuclease III digestion, the combined aliquots are treated with mung bean nuclease to remove the resulting 5′ overhang. The termini are then polished with T4 DNA polymerase, and the plasmid is recircularized with T4 DNA ligase to produce the deletion library. The deletion library is then converted into a fragment library (14 base-pair fragments in this case) by digestion with restriction endonucleases BsmI and BpmI, purification of the plasmid containing the 14 bp fragment from the excised BpmI/BsmI fragment, end-polishing with T4 DNA polymerase, and ligation with T4 DNA ligase. After each ligation step, the ligation mixture is transformed into bacteria, the DNA is recovered from the bacteria, and the recovered DNA is used in the subsequent step.

All of these reactions involving restriction endonucleases, ligases, polymerases, nucleases, and the like are well known in the art and are performed according to standard methods, e.g., Sambrook et al., Molecular Cloning: A Laboratory Manual, (2d ed., 1989); Maniatis et al., Molecular Cloning: A Laboratory Manual, (1982); Ausubel et al., Current Protocols in Molecular Biology, (1987), relevant parts of which are hereby incorporated by reference.

Other types of antisense libraries can also be produced from the fragment library. For instance, other cassettes can be ligated into an HphI-digested fragment library. Catalytic cores from ribozymes can be inserted. Alternatively, cassettes may be used that encode sequences that silence the target by mechanisms other than cleavage. Similarly, ribozyme and non-ribozyme sequences can be added to the end of the antisense sequence. In one embodiment, the DNA fragment library is digested with BpmI, which digests the DNA at the distal end of the inserted fragment. The unpaired nucleotides resulting from this reaction are then removed with T4 DNA polymerase to result in blunt ends. Next, a cassette is inserted by ligation to recircularize the modified plasmid, which now contains the cassette inserted at an end of the insert fragment. Alternatively, instead of inserting a cassette after the fragment library is produced, a suitable cassette can be engineered into the starting multi-cloning sequence.

Antisense libraries prepared according to the present invention can be assayed in vitro in a cell free system or in vivo in cultured cells to select effective antisense agents. In vivo, the antisense library is introduced by transfection into a suitable cell line that expresses the gene of interest. The term “transfection” is used herein to refer to the uptake of foreign DNA by a cell. Thus, a cell has been “transfected” when exogenous DNA has been introduced inside the cell membrane. A number of transfection techniques are generally known in the art. See, e.g., Graham et al., Virology, 52:456 (1973); Sambrook et al., Molecular Cloning, A Laboratory Manual, Cold Spring Harbor Laboratories, (1989); Davis et al., Basic Methods in Molecular Biology, (1986), and Chu et al., Gene, 13:197 (1981). Such techniques can be used to introduce one or more exogenous DNA moieties, such as a nucleotide integration vector and other nucleic acid molecules, into suitable host cells.

“Host cells” may be either eukaryotic or prokaryotic. In particular, host cells could be yeast cells, insect cells, or mammalian cells that have been transfected with an exogenous DNA sequence, as well as the progeny of those cells. It is understood that the progeny of a single parental cell may not necessarily be completely identical in morphology or in genomic or total DNA complement as the original parent, due to natural, accidental, or deliberate mutation.

One of skill in the art may appreciate that transfection conditions may be chosen such that generally only one member of the library is taken up by each individual host cell. The individual cells then each express a different antisense molecule targeted to a different site on the RNA transcript of interest. All target sites are represented in the entire cell population produced by transfection. Using a suitable detection method, cell clones can be identified by DNA sequencing.

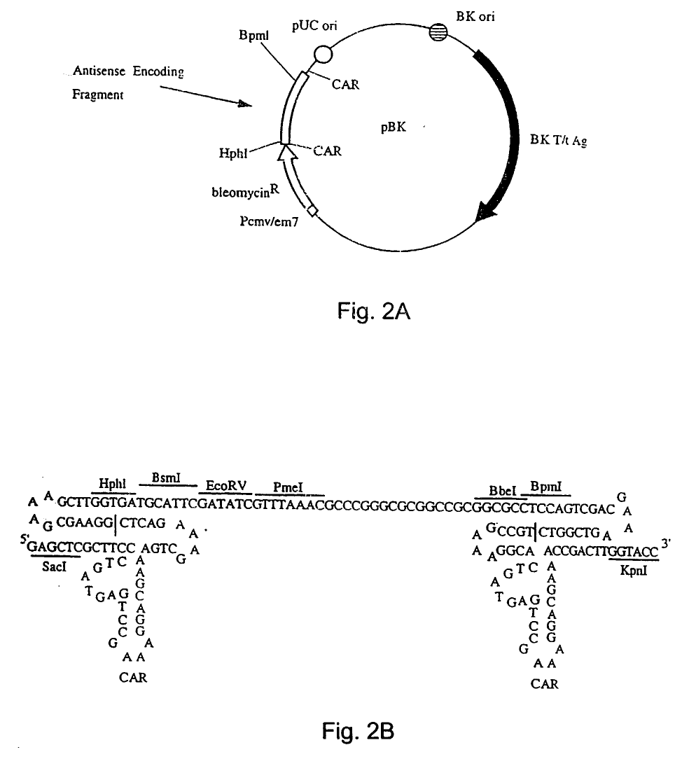

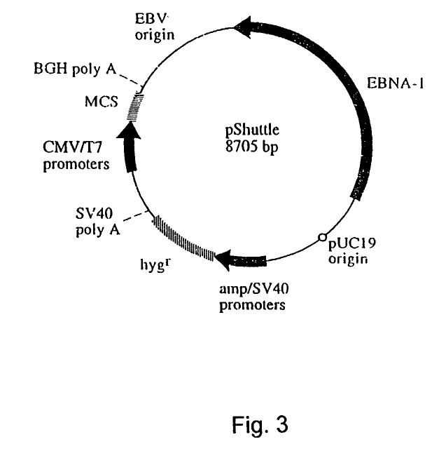

To identify suitable targets in vivo, specially designed expression vectors are required. Such expression vectors may in one embodiment be designed to replicate episomally in mammalian cells. pBK and pShuttle are two such vectors. As shown in FIG. 2A, vector pBK (SEQ ID NO: 14) possesses the origin of replication and the gene encoding the T/t antigen from the human papova virus BK (BKV). As seen in FIG. 3, vector pShuttle (SEQ ID NO: 16) possesses the origin of replication and the EBNA1 gene from the human Epstein-Barr virus (EBV). These sequence elements allow each of the plasmids to replicate extrachromosomally (episomally).

Vector pBK illustrates other features of value for in vivo expression of antisense libraries and may be used to produce oligonucleotides according to the invention. PBK has a single antibiotic resistance gene, bleomycin^{R}, driven by dual mammalian (CMV) and bacterial (em7) promoters. This allows the same selectable marker to be used in both bacterial and mammalian cells, and can be shuttled between them. PBK was designed such that the antisense library could be constructed and expressed from the same vector. The antisense sequence is expressed by read-through expression of the bleomycin gene. This ensures expression of the antisense agent when the cells are grown in the presence of bleomycin.

The antisense fragment is released from the larger bleomycin transcript by the activity of cisacting ribozymes (CAR), hammerhead ribozymes in this case, that flank the antisense sequence. In the absence of CAR, flanking sequences of the larger bleomycin transcript could inhibit the activity of the antisense agent. Sequences outside of the MCS encode the cis-acting ribozymes. This is illustrated in FIG. 2B, where only the hairpin-terminal oligonucleotide is shown (SEQ ID NO: 15). On cleavage by the CAR, the oligonucleotide agent is released and stable hairpin loops form to increase the nuclease resistance of the gene silencing agent.

Although it is believed that episomal shuttle vectors are advantageous for expression of directed antisense libraries, viral vectors can also be used. Many viruses are currently being examined for expression of foreign genes for the purpose of gene therapy. These same viral vectors would be suitable for expression of directed antisense libraries. Some of these vectors replicate extrachromosomally and therefore behave similarly to the described episomal vectors. Others integrate into chromosomes. For the use of integrative viral vectors, two minor problems would need to be dealt with. First, the antisense gene present within the viral vector would integrate into the chromosome with the virus. Consequently, recovering the gene to determine the site at which it targets is not readily possible. This can be dealt with by using polymerase chain reaction (PCR) to amplify the integrated antisense gene. The PCR product could be sequenced directly, or cloned and sequenced to identify the target site. Second, some of these viral vectors integrated randomly and this would produce differing levels of expression from different members of the directed antisense library. As discussed, it is important that expression of all members of the library be comparable. This problem can be dealt with by using a viral vector that integrates at a specific preferred site, such as adeno-associated virus.

In vitro assays can also be used to identify effective antisense targets. Lieber & Strauss, Molecular and Cellular Biology, 15:540-551 (1995). In such assays, the antisense library is produced by in vitro transcription from a suitable promoter. In the present case, an antisense ribozyme library in pShuttle (SEQ ID NO: 16) might be used. Of course other types of antisense libraries could be used similarly. The library-containing pShuttle is digested with XbaI and used as a template for run-off transcription of the antisense ribozyme by in vitro transcription with T7 RNA polymerase, according to methods well known in the art. See, e.g., Noren et al, Nucleic Acids Res., 18:83-88 (1990). Subsequently, the transcribed ribozyme library is incubated in a lysate prepared from a mammalian cell line expressing the gene of interest. Effective target sites are identified by performing a primer extension reaction on purified RNA from the lysate using a primer specific for the gene of interest. Primer extension products terminate at the sites of cleavage by effective ribozymes. These sites are identified by gel electrophoresis of the primer extension products with suitable size markers.

In other embodiments of the invention, the targeting sequence is selected without regard to its antisense properties. Instead, the targeting sequence may be selected for its effectiveness when used as a siRNA molecule. siRNA sequences are generally from about 21 nucleotides to about 23 nucleotides in length. These molecules are generally paired such that they have a two-nucleotide 3′ overhang. The sequence of the siRNA may essentially be selected randomly from within a target sequence of a selected nucleic acid. Tuschl et al., The siRNA user guide, http://www.mpibpc.gwdg.de/abteilungen/100/105/sirna.html, revised Jul. 12, 2002. Target sequences are selected on a specified nucleic acid molecule generally 50 to 100 nucleotides downstream of the start codon. Id.

In addition to the targeting sequence discussed above, the oligonucleotides of the invention further include either a pair of hairpin loop oligonucleotides coupled to the 5′ and 3′ ends of the targeting region, or a single 3′ or 5′ hairpin loop. The hairpin loops of the oligonucleotides generally include stem regions and loop regions, and are positioned on the 3′ and 5′ ends of the targeting sequence. The stem region of the hairpin loop oligonucleotide is composed of a set of nucleotides capable of stably pairing which are separated by a region that becomes the loop region of the oligonucleotide when the oligonucleotide has obtained its final conformation. These stem sequences are generally inverted complementary repeats separated from each other by the loop region.

The stem region includes a set of from about 1 to about 12 paired nucleotides. More preferably, the stem region includes from about 2 to about 10 paired nucleotides. Still more preferably, the stem region includes from about 4 to about 6 paired nucleotides. It is further preferred that the loop region of the oligonucleotides include from at least about 1 to at least about 10 unpaired nucleotides. More preferably, the loop region includes from about 2 to about 8 unpaired nucleotides. Still more preferably, the loop region includes from about 4 to about 6 unpaired nucleotides.

In some embodiments of the oligonucleotides of the invention, the 3′ and 5′ hairpin loop sequences are attached to the targeting sequence by linker sequences of from about 1 to about 12 nucleotides in length. More preferably, these linker sequences are from about 2 to about 8 nucleotides in length. Still more preferably, these linker sequences are from about 4 to about 6 nucleotides in length.

The oligonucleotides of the invention have been shown to effectively knock down the production of the product of the selected gene when administered to a cell containing the selected gene. Without being limited to any one theory, it appears that the hairpin-terminal oligonucleotides of the invention cause gene silencing at least in part using RNA interference mechanisms. RNA interference is an inhibitive process generally sparked by the introduction of a double-stranded RNA (“dsRNA”) to a cell. These dsRNAs are generally cleaved into 21-23 nucleotide segments by an enzyme dubbed DICER. Following this process, the oligonucleotides are recognized and bound by a nuclease complex forming a complex referred to as a small interfering ribonucleoprotein particle (“siRNP”) which then proceeds to seek out oligonucleotides having a sequence complementary to the sequence of the bound dsRNA fragment. Those mRNAs present with the specific sequence are targeted and destroyed, knocking down the expression of the gene product in the cell. As discussed in the examples below, the oligonucleotides of the invention have been shown to be effective in bringing about effective gene product knockdown using RNA oligonucleotides.

In addition, the terminal-hairpin oligonucleotides of the invention which have an antisense targeting sequence may function in an antisense manner by directly interfering with the translation of complementary mRNA molecules located in vivo. The terminal hairpin loop or loops of the oligonucleotide may add to the function in this mechanism by helping to stabilize the oligonucleotide in the presence of cellular nucleases.

Any of the compounds of the present invention can be synthesized as pharmaceutically acceptable salts for incorporation into various pharmaceutical compositions. The term “pharmaceutically acceptable salts” refers to salts of the compounds of the invention which are substantially non-toxic to living organisms. Typical pharmaceutically acceptable salts include those salts prepared by reaction of the compounds of the invention with a pharmaceutically acceptable mineral or organic acid, or a pharmaceutically acceptable alkali metal or organic base, depending on the substituents present on the compounds of the formulae.

Examples of pharmaceutically acceptable mineral acids which may be used to prepare pharmaceutically acceptable salts include hydrochloric acid, phosphoric acid, sulfuric acid, hydrobromic acid, hydroiodic acid, phosphorous acid and the like. Examples of pharmaceutically acceptable organic acids which may be used to prepare pharmaceutically acceptable salts include aliphatic mono and dicarboxylic acids, such as oxalic acid, carbonic acid, citric acid, succinic acid, phenyl-substituted alkanoic acids, aliphatic and aromatic sulfuric acids and the like. Such pharmaceutically acceptable salts prepared from mineral or organic acids thus include hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydrofluoride, acetate, propionate, formate, oxalate, citrate, lactate, p-toluenesulfonate, methanesulfonate, maleate, and the like.

It should be recognized that the particular anion or cation forming a part of any salt of this invention is not critical, so long as the salt, as a whole, is pharmacologically acceptable and as long as the anion or cationic moiety does not contribute undesired qualities. Further, additional pharmaceutically acceptable salts are known to those skilled in the art.

Additionally, the compounds of the invention may be combined with a pharmaceutically acceptable carrier to provide pharmaceutical compositions for treating biological conditions or disorders such as those briefly noted herein in organisms such as mammalian patients, and more preferably, in human patients. The particular carrier employed in these pharmaceutical compositions may take a wide variety of forms depending upon the type of administration desired, e.g., intravenous, oral, topical, suppository or parenteral. In some configurations of the invention, the oligonucleotides of the invention may be utilized in a chemically-modified form, or with a carrier such as the copolymers taught in U.S. patent application Ser. No. 09/647,344.

In preparing the compositions in oral liquid dosage forms (e.g., suspensions, elixirs and solutions), typical pharmaceutical media, such as water, glycols, oils, alcohols, flavoring agents, preservatives, coloring agents and the like can be employed. Similarly, when preparing oral solid dosage forms (e.g., powders, tablets and capsules), carriers such as starches, sugars, diluents, granulating agents, lubricants, binders, disintegrating agents and the like will be employed. Due to their ease of administration, tablets and capsules represent the most advantageous oral dosage form for the pharmaceutical compositions of the present invention.

For parenteral administration, the carrier will typically comprise sterile water, although other ingredients that aid in solubility or serve as preservatives, may also be included. Furthermore, injectable suspensions may also be prepared, in which case appropriate liquid carriers, suspending agents and the like will be employed.

For topical administration, the compounds of the present invention may be formulated using bland, moisturizing bases, such as ointments or creams. Examples of suitable ointment bases are petrolatum, petrolatum plus volatile silicones, lanolin, and water in oil emulsions.

As recognized by those skilled in the art, the particular quantity of pharmaceutical composition according to the present invention administered to a patient will depend upon a number of factors, including, without limitation, the biological activity desired, the condition of the patient, and tolerance for the drug.

Specific embodiments of the oligonucleotides are discussed in the Examples below. These examples depict only typical embodiments of the invention, and are not to be considered to be limiting of its scope.

### EXAMPLES

### Example 1

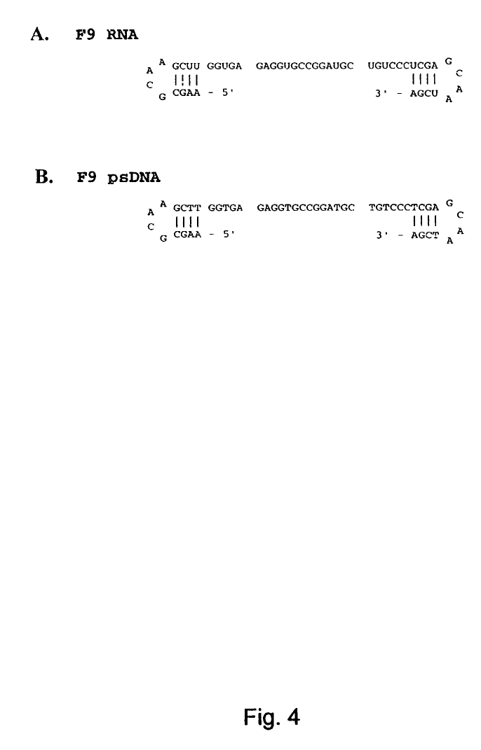

In a first example, the oligonucleotides of FIGS. 4A (SEQ ID NO: 1) and 4B (SEQ ID NO: 2) were tested for their ability to silence the expression of MMP-9 in vivo. As discussed above, the F9 RNA of FIG. 4A is an all-RNA oligonucleotide targeted against MMP-9 mRNA by its central 14-nucleotide targeting sequence. The F9 psDNA of FIG. 4B is identical to F9 RNA except that it is composed of deoxynucleotides instead of ribonucleotides. Specifically, thymidines replace the uridines found in F9 RNA, and in addition, phosphorothioate linkages replace all of the phosphodiester linkages found in the F9 RNA.

The F9 RNA of FIG. 4A (SEQ ID NO: 1) and the F9 psDNA of FIG. 4B (SEQ ID NO: 2) both include 5′ and 3′ terminal hairpin loops, each having a stem of four sets of paired nucleotides and a loop of four unpaired nucleotides. Further, as noted, in these oligonucleotides, the hairpin loops are linked to the targeting sequence by linker sequences. In these specific oligonucleotides, the linker sequences are 5 nucleotides long on the 5′ end and 6 nucleotides long on the 3′ end.

In this first Example, these oligonucleotides were used to treat HT1080 cells. A first set of HT1080 cells received no oligonucleotide, and thus acted as a control. Second and third sets received F9 RNA or F9 psDNA, respectively. The oligonucleotides were added to the media of the HT1080 cell culture at a concentration of 1 micromolar in the presence of a copolymer having characteristics detailed in U.S. patent application Ser. No. 09/647,344, as well as commercially-available transfection reagents. Following this, the cells were cultured in the presence of the oligonucleotide for 21.5 hours. The cells were subsequently harvested and polyA mRNA was isolated using the PolyA Tract System 1000 (Promega, Inc, Madison, Wis.).

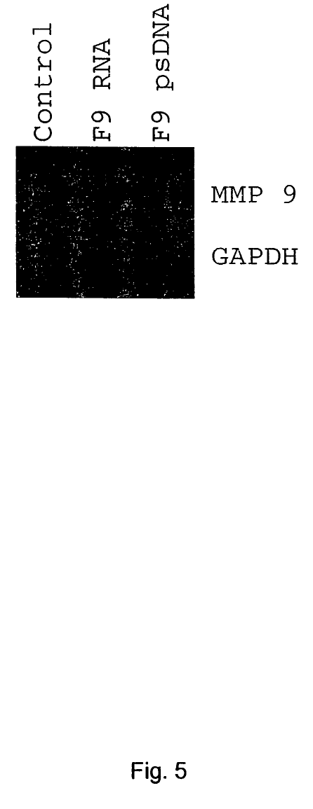

MMP-9 mRNA expression levels were examined by RT-PCR as described in Wong, et al., Monitoring MMP and TIMP mRNA expression by RT-PCR, Methods Mol. Biol., 151:305-20 (2001). According to this method, the polyA mRNA obtained above was reverse transcribed using AMV reverse transcriptase according to the manufacturer's instructions (Promega, Inc, Madison, Wis.). The polyA mRNA was then subjected to PCR amplification using Taq DNA polymerase (Promega, Inc, Madison, Wis.). The PCR amplification was conducted using PCR primers specific to MMP-9 and to glyceraldehyde phosphate dehydrogenase (GAPDH). The PCR reactions were then loaded onto 1.8% agarose gels, which were then electrophoresced. The gels were then stained with ethidium bromide to yield the gel shown in FIG. 5.

FIG. 5 is a photograph of the gel containing the results of the PCR amplification of the polyA obtained from the cell culture without oligonucleotides, the culture exposed to F9 RNA (SEQ ID NO: 1), and the culture exposed to F9 psDNA (SEQ ID NO: 2), respectfully, as labeled along the upper axis of the gel. The MMP-9 and GAPDH-specific PCR fragments are designated along the vertical axis of the gel.

The gel shows no inhibition of MMP-9 mRNA expression in the control sample, while in the sample exposed to F9 RNA (SEQ ID NO: 1), expression of MMP-9 was nearly completely inhibited. With respect to the sample exposed to F9 psDNA (SEQ ID NO: 2), much less inhibition was observed.

### Example 2

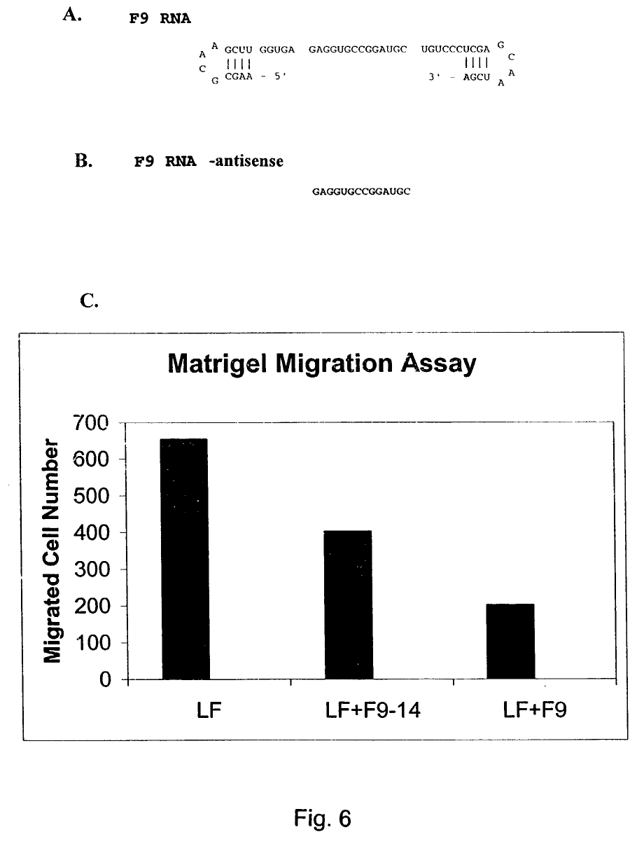

In a second example, the efficacy of the oligonucleotides of FIGS. 6A (SEQ ID NO: 1) and 6B (SEQ ID NO: 3) was tested in an in vitro model. FIG. 6A shows the F9 RNA oligonucleotide with terminal hairpins used in Example 1. FIG. 6B shows a 14-nucleotide antisense F9 RNA oligonucleotide such as is commonly used in antisense applications. Both of these oligonucleotides are synthetic molecules.

These two molecules were screened in vitro for their ability to inhibit cell invasion in Transwell Matrigel filters. The Matrigel invasion assay is a standard assay representative of the in vivo invasion of the lining of blood vessels by cancer cells and activated T cells. In this assay, about 250,000 HT1080 human fibroblast sarcoma cells cultured in 1 ml EMEM+10% FBS were plated in 6 wells of a 12 well plate and incubated overnight at 37° C. in 5% CO₂. Following incubation, the plates were washed with DPBS. Next, 400 μl of serum-free EMEM media was added to the plates. Control wells and testing wells each received equal amounts of Lipofectamine solution (Invitrogen). The control wells received only the transfection agent Lipofectamine 2000. The testing wells received a solution of Lipofectamine 2000 and 1 μM F9 RNA or 1 μM F9 RNA with terminal hairpins.

After the addition of the transfection solutions, the samples were incubated for 6 hours at 37° C. in 5% CO₂. Following this, the media were aspirated and replaced with EMEM media containing 10% FBS. The samples were then incubated for another 48 hours at 37° C. in 5% CO₂ .

Following incubation, about 100,000 cells from each sample were used in a Matrigel invasion assay. 100,000 cells were plated per Transwell in a 500 μl volume of serum containing EMEM media. These cells were cultured for 16-20 hours at 37° C. in 5% CO₂. Following incubation, the cells were fixed by immersing the Transwell filters in methanol for 15 min. The Transwell filters were next washed once with water, stained in 0.1% toluidine blue for 5 minutes, and then washed until cell staining became visible.

Cells on the upper surface of the membrane were removed with a cotton swab. Those cells that had migrated to the underside of the membrane were counted microscopically. The results of the Matrigel assay are presented in the table of FIG. 6C. As indicated in the table, at a concentration of 1 μM, the 14-nucleotide antisense RNA inhibited 40% of HT1080 cell migration compared to the control cells treated with the transfection agent Lipofectamine 2000 alone. In contrast, those cells treated with the F9 RNA oligonucleotide with terminal hairpins showed 70% inhibition of HT1080 cell migration. This assay demonstrates the superior ability of the oligonucleotides having terminal hairpin loops in preventing expression of MMP-9, thus substantially blocking cell migration.

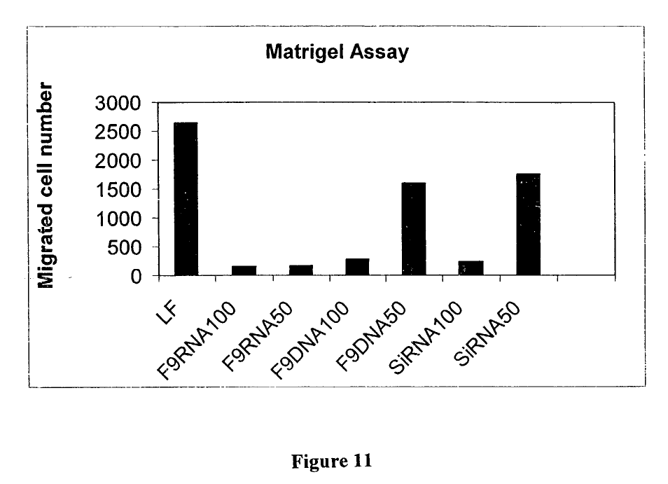

The results of a similar invasion assay are included in FIG. 11. In this assay, the efficacy of the F9 RNA was compared with that of phosphorothioate DNA and siRNAs. In all cases, the F9 RNA proved more effective in preventing migration.

### Example 3

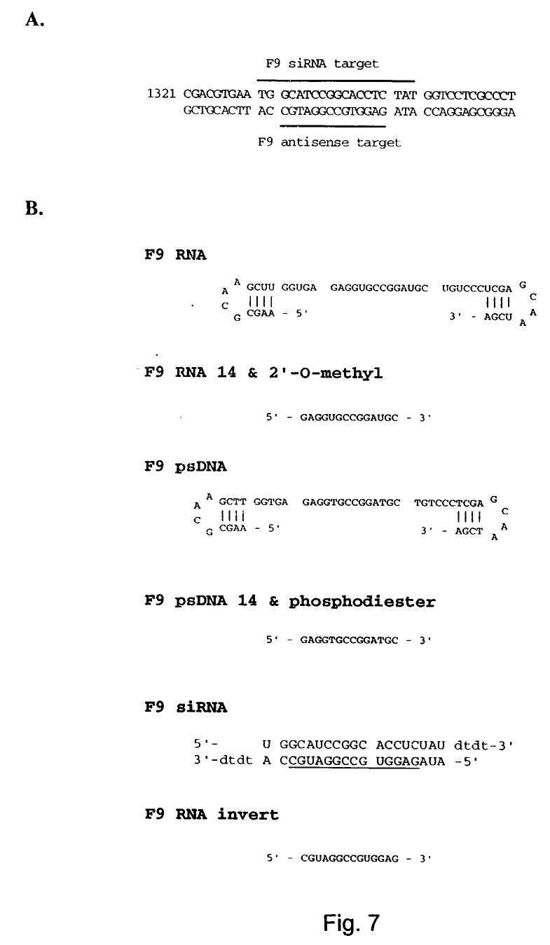

In a third Example, HT1080 cells were treated with a variety of oligonucleotides directed against the F9 target site of MMP-9 mRNA. This target site is shown in FIG. 7A, which indicates both the antisense (SEQ ID NO: 5) and siRNA (SEQ ID NO: 4) target sites. This assay allows further evaluation of the efficacy of the terminal-hairpin oligonucleotides of the invention in comparison with other oligonucleotides used in other gene-silencing technologies such as antisense and RNA interference. The oligonucleotides used are shown in FIG. 7B.

The first oligonucleotide shown is the F9 RNA oligonucleotide (SEQ ID NO: 1) with terminal hairpins of the invention. This oligonucleotide is an all RNA oligonucleotide comprised of a central 14-base targeting sequence that targets the F9 antisense target site connected to 5′ and 3′ terminal hairpin loops by 5- and 6-base single-stranded non-complementary linker sequences, respectively. The second oligonucleotide is an RNA antisense oligonucleotide with 2′-O-methyl linkages (SEQ ID NO: 6). The third oligonucleotide is a F9 psDNA (SEQ ID NO: 2) having an identical targeting sequence to the F9 RNA with terminal hairpins which has a phosphorothioate DNA backbone instead of the RNA backbone of the F9 RNA oligonucleotide. The fourth oligonucleotide is a F9 psDNA 14 & phosphodiester oligonucleotide (SEQ ID NO: 7).

These first four oligonucleotides each contain a substantially similar 14-base targeting sequence targeting the oligonucleotide to the F9 antisense target site, albeit possibly through a variety of mechanisms. These oligonucleotides differ, however, in the composition of their backbones, RNA versus 2′-O-methyl, phosphorothioate DNA and phosphodiester DNA, respectively.

The next oligonucleotide is an oligonucleotide configured to silence the MMP-9 gene using RNA-interference methods (SEQ ID NO: 8, SEQ ID NO: 9). This F9-targeted siRNA is a double-stranded RNA duplex that targets the F9 siRNA target site. The F9 siRNA target sequence is 19 nucleotides long instead of 14 nucleotides long. This longer sequence is reported to be optimal for siRNA. The final oligonucleotide is a F9 RNA invert (SEQ ID NO: 10) used as a control. This oligonucleotide includes an RNA backbone but uses the F9 antisense target sequence encoded in an inverted, and thus non-complementary, orientation.

In this assay, HT1080 cells were plated in 6-well culture-plates at a density of 300,000 to 500,000 cells per well. Following plating, the cell culture media was removed from the wells and replaced with serum-free media (EMEM). Each of the oligonucleotides was complexed with Lipofectamine 2000 (Invitrogen), as per the manufacturer's instructions, using 20 microliters of Lipofectamine per nanomole of oligonucleotide.

The complexed oligonucleotides were added to the cells at a final concentration of 1 μM for all except the siRNA, which was added at a final concentration of 0.25 μM. As an added control, an equivalent amount of LF was added to one well in the absence of any oligonucleotide. The cells were cultured for 6 hours, after which the media was removed and replaced with serum-containing EMEM. The cells were then cultured for an additional 42 hours, after which the cells were harvested.

PolyA mRNA was isolated from the harvested cells using the PolyA Tract System 1000 (Promega, Inc, Madison, Wis.). MMP-9 mRNA expression levels were examined by RT-PCR as described in Wong et al., Monitoring MMP and TIMP mRNA expression by RT-PCR, Methods Mol. Biol., 151:305-20 (2001). According to this method, polyA mRNA was reverse-transcribed using AMV reverse transcriptase according to the manufacturer's instructions (Promega, Inc, Madison, Wis.). The polyA mRNA was then subjected to PCR amplification using Taq DNA polymerase (Promega, Inc, Madison, Wis.). The PCR amplification was conducted using PCR primers specific to MMP-9 and to glyceraldehyde phosphate dehydrogenase (GAPDH).

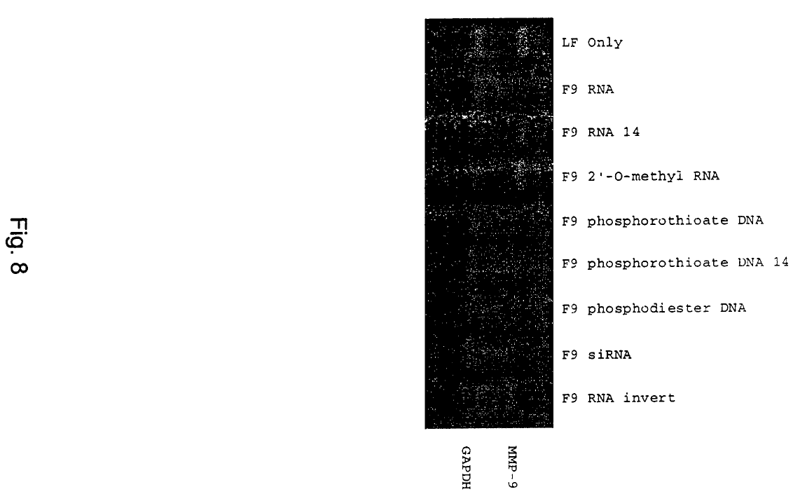

The products of the PCR reactions were then loaded onto 1.8% agarose gels, which were electrophoresced. The gels were then stained with ethidium bromide, producing the gel shown in FIG. 8. The MMP-9 and GAPDH-specific PCR fragments are designated along the right vertical axis of the gel, and the oligonucleotides used in each sample are designated along the horizontal axis of the gel in alignment with the associated lane on the gel.

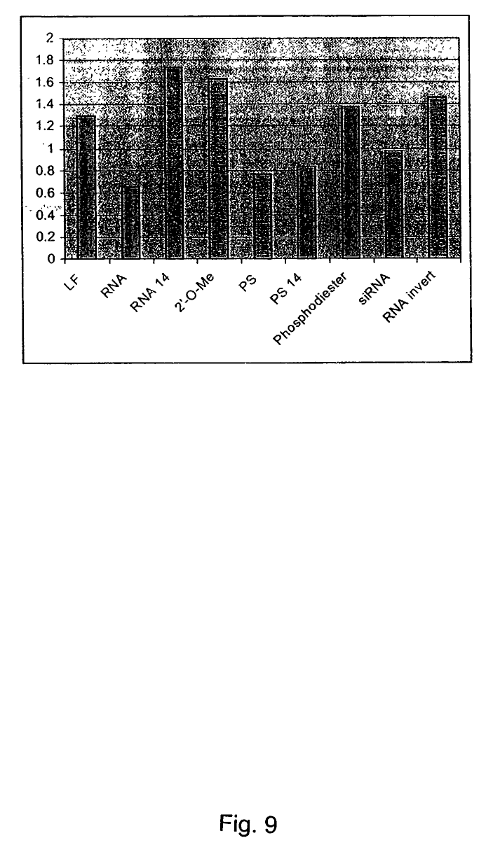

The intensity of each of the bands seen on the gel was measured using the NIH image computer image processing and analysis program. The ratio of the intensities of MMP-9 to GAPDH was determined for each lane and plotted in the table of FIG. 9.

As is visible in FIG. 9, MMP-9 mRNA expression is significantly inhibited, relative to GAPDH, by the F9 RNA oligonucleotides with terminal hairpins (SEQ ID NO: 1), the phosphorothioate DNA oligonucleotides with terminal hairpins (SEQ ID,NO: 2), the 14-nucleotide phosphorothioate DNA antisense oligonucleotide (SEQ ID NO 7), and to a lesser extent by the F9-targeted siRNA oligonucleotide (SEQ ID NO: 8, SEQ ID NO: 9). The control RNA invert (SEQ ID NO: 10) and remaining oligonucleotides are no more effective than Lipofectamine 2000 alone.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

What is claimed and desired to be secured by United States Letters Patent is:

1. A compound for silencing a gene comprising:
a single-stranded nucleic acid molecule having a 3′ end, a 5′ end, and a targeting region positioned between the 3′ end and the 5′ end;
wherein the targeting region comprises a sequence targeted to a target region in the gene; and
wherein the 3′ end and the 5′ end each comprise a sequence that enables the formation of a hairpin structure.

2. The compound for silencing a gene of claim 1, wherein the targeting region of the single-stranded nucleic acid molecule is between about 8 and about 50 nucleotides in length.

3. The compound for silencing a gene of claim 2, wherein the targeting region of the single-stranded nucleic acid molecule is between about 12 and about 20 nucleotides in length.

4. The compound for silencing a gene of claim 3, wherein the targeting region of the single-stranded nucleic acid molecule is about 14 nucleotides in length.

5. The compound for silencing a gene of claim 3, wherein the targeting region of the single-stranded nucleic acid molecule is about 18 nucleotides in length.

6. The compound for silencing a gene of claim 1, wherein the targeting region of the single-stranded nucleic acid molecule is substantially identical to a target region in the gene.

7. The compound for silencing a gene of claim 1, wherein the targeting region of the single-stranded nucleic acid molecule is substantially complementary to a target region in the gene.

8. The compound for silencing a gene of claim 1, wherein the single-stranded nucleic acid molecule is a RNA molecule.

9. The compound for silencing a gene of claim 1, wherein the single-stranded nucleic acid molecule is a DNA molecule.

10. The compound for silencing a gene of claim 1, wherein the single-stranded nucleic acid molecule includes nucleotide analogs.

11. The compound for silencing a gene of claim 10, wherein the nucleotide analogs are selected from the group consisting of phosphorothioates, 2′O-methyl analogs, 2′O-amino analogs, and 2′O-fluoro analogs.

12. The compound for silencing a gene of claim 1, wherein the single-stranded nucleic acid molecule comprises mixed-backbone linkages.

13. The compound for silencing a gene of claim 1, wherein the single-stranded nucleic acid molecule is from about 14 to about 114 nucleotides in length.

14. The compound for silencing a gene of claim 13, wherein the single-stranded nucleic acid molecule is from about 14 to about 72 nucleotides in length.

15. The compound for silencing a gene of claim 14, wherein the single-stranded nucleic acid molecule is about 54 nucleotides in length.

16. The compound for silencing a gene of claim 15, wherein the single-stranded nucleic acid molecule is about 50 nucleotides in length.

17. The compound for silencing a gene of claim 1, wherein the single-stranded nucleic acid molecule further comprises a linker sequence positioned between the targeting region and the 5′ end.

18. The compound for silencing a gene of claim 1, wherein the single-stranded nucleic acid molecule further comprises a linker sequence positioned between the targeting region and the 3′ end.

19. The compound for silencing a gene of claim 1, wherein the single-stranded nucleic acid molecule further comprises linker sequences positioned between the targeting region and both the 5′ end and the 3′ end.

20. The compound for silencing a gene of claim 19, wherein the linker sequences include between about 1 and about 10 nucleotides.

21. The compound for silencing a gene of claim 19, wherein the linker sequences include between about 4 and about 8 nucleotides.

22. The compound for silencing a gene of claim 19, wherein the linker sequences include about 6 nucleotides.

23. The compound for silencing a gene of claim 1, wherein the sequence that enables the formation of a hairpin structure includes inverted repeats.

24. The compound for silencing a gene of claim 1, wherein the sequence that enables the formation of a hairpin structure comprises a loop region including between about 1 and about 10 nucleotides.

25. The compound for silencing a gene of claim 24, wherein the sequence that enables the formation of a hairpin structure comprises a loop region including between about 2 and about 8 nucleotides.

26. The compound for silencing a gene of claim 25, wherein the sequence that enables the formation of a hairpin structure comprises a loop region including between about 4 to about 6 nucleotides.

27. A recombinant vector comprising a nucleic acid encoding the compound for silencing a gene of claim 1.

28. The recombinant vector of claim 27, wherein the recombinant vector is a plasmid.

29. The recombinant vector of claim 27, wherein the recombinant vector is a prokaryotic or eukaryotic expression vector.

30. The recombinant vector of claim 27, wherein the nucleic acid encoding the compound for silencing a gene is operably linked to a heterologous promoter.

31. A host cell comprising the nucleic acid of claim 1.

32. The host cell of claim 31, wherein the host cell is a eukaryotic host cell.

33. The host cell of claim 31, wherein the host cell is a prokaryotic host cell.

34. An oligonucleotide having a structure represented by:
H²-R¹-H¹
wherein R¹ is an oligonucleotide consisting essentially of RNA of between about 8 and about 50 nucleotides configured to silence a substrate nucleic acid, and wherein H¹ and H² are oligonucleotides having sequences that enable the formation of a hairpin structure.

35. The oligonucleotide of claim 34, wherein R¹ is between about 12 and about 20 nucleotides in length.

36. The oligonucleotide of claim 35, wherein R¹ is about 14 nucleotides in length.

37. The oligonucleotide of claim 35, wherein R¹ is about 18 nucleotides in length.

38. The oligonucleotide of claim 34, wherein R¹ is substantially complementary to at least a portion of the substrate nucleic acid.

39. The oligonucleotide of claim 34, wherein R¹ is substantially identical to at least a portion of the substrate nucleic acid.

40. The oligonucleotide of claim 34, wherein the H¹ and H² oligonucleotides have a length of less than about 30 nucleotides.

41. The oligonucleotide of claim 40, wherein the H¹ and H² oligonucleotides have a length of from about 4 to about 24 nucleotides.

42. The oligonucleotide of claim 41, wherein the H¹ and H² oligonucleotides have a length of from about 8 to about 20 nucleotides.

43. The oligonucleotide of claim 42, wherein the H¹ and H² oligonucleotides have a length of from about 10 to about 16 nucleotides.

44. An oligonucleotide having a structure represented by:
H²-R¹-H¹
wherein R¹ is an oligonucleotide consisting essentially of DNA of between about 8 and about 50 nucleotides configured to silence a substrate nucleic acid, and wherein H¹ and H² are oligonucleotides having sequences that enable the formation of a hairpin structure.

45. The oligonucleotide of claim 44, wherein R¹ is between about 12 and about 20 nucleotides in length.

46. The oligonucleotide of claim 45, wherein R¹ is about 14 nucleotides in length.

47. The oligonucleotide of claim 45, wherein R¹ is about 18 nucleotides in length.

48. The oligonucleotide of claim 44, wherein R¹ is substantially complementary to at least a portion of the substrate nucleic acid.

49. The oligonucleotide of claim 44, wherein R¹ is substantially identical to at least a portion of the substrate nucleic acid.

50. The oligonucleotide of claim 44, wherein the H¹ and H² oligonucleotides have a length of less than about 30 nucleotides.

51. The oligonucleotide of claim 50, wherein the H¹ and H² oligonucleotides have a length of from about 4 to about 24 nucleotides.

52. The oligonucleotide of claim 51, wherein the H¹ and H² oligonucleotides have a length of from about 8 to about 20 nucleotides.

53. The oligonucleotide of claim 52, wherein the H¹ and H² oligonucleotides have a length of from about 10 to about 16 nucleotides.

54. A pharmaceutical composition for silencing a gene, the composition comprising:
a pharmaceutically acceptable carrier; and
a single-stranded nucleic acid molecule having a 3′ end, a 5′ end, and a targeting region positioned between the 3′ end and the 5′ end, wherein the targeting region has a sequence targeted to a target region in the gene, and wherein the 3′ end and the 5′ end each have a sequence that enables a formation of a hairpin structure.

55. The pharmaceutical composition for silencing a gene of claim 54, wherein the targeting region of the single-stranded nucleic acid molecule is between about 8 and about 50 bases in length.

56. The pharmaceutical composition for silencing a gene of claim 55, wherein the targeting region of the single-stranded nucleic acid molecule is between about 12 and about 20 bases in length.

57. The pharmaceutical composition for silencing a gene of claim 56, wherein the targeting region of the single-stranded nucleic acid molecule is about 14 bases in length.

58. The pharmaceutical composition for silencing a gene of claim 56, wherein the targeting region of the single-stranded nucleic acid molecule is about 18 bases in length.

59. The pharmaceutical composition for silencing a gene of claim 54, wherein the targeting region of the single-stranded nucleic acid molecule is substantially identical to a target region in the gene.

60. The pharmaceutical composition for silencing a gene of claim 54, wherein the targeting region of the single-stranded nucleic acid molecule is substantially complementary to a target region in the gene.

61. The pharmaceutical composition for silencing a gene of claim 54, wherein the single-stranded nucleic acid molecule comprises RNA.

62. The pharmaceutical composition for silencing a gene of claim 54, wherein the single-stranded nucleic acid molecule comprises DNA.

63. The pharmaceutical composition for silencing a gene of claim 54, wherein the single-stranded nucleic acid molecule includes nucleotide analogs.

64. The pharmaceutical composition for silencing a gene of claim 63, wherein the nucleotide analogs are selected from the group consisting of phosphorothioates, 2′O-methyl analogs, 2′O-amino analogs, and 2′O-fluoro analogs.

65. The pharmaceutical composition for silencing a gene of claim 54, wherein the single-stranded nucleic acid molecule further comprises a linker sequence positioned between the targeting region and the 5′ end.

66. The pharmaceutical composition for silencing a gene of claim 54, wherein the single-stranded nucleic acid molecule further comprises a linker sequence positioned between the targeting region and the 3′ end.

67. The pharmaceutical composition for silencing a gene of claim 54, wherein the single-stranded nucleic acid molecule further comprises linker sequences positioned between the targeting region and both the 5′ end and the 3′ end.

68. The pharmaceutical composition for silencing a gene of claim 67, wherein the linker sequences include between about 1 and about 10 nucleotides.

69. The pharmaceutical composition for silencing a gene of claim 67, wherein the linker sequence includes between about 4 and about 8 bases.

70. The pharmaceutical composition for silencing a gene of claim 67, wherein the linker sequence includes about 6 bases.

71. The pharmaceutical composition for silencing a gene of claim 54, wherein the sequence that enables the formation of a hairpin structure includes inverted repeats.

72. The compound for silencing a gene of claim 54, wherein the sequence that enables the formation of a hairpin structure includes between about 3 and about 30 bases.

73. The compound for silencing a gene of claim 71, wherein the sequence that enables the formation of a hairpin structure includes between about 6 and about 20 bases.

74. The compound for silencing a gene of claim 72, wherein the sequence that enables the formation of a hairpin structure includes from about 8 to about 18 bases.

75. A method of silencing a gene in a cell comprising the steps of:
contacting the cell with a compound comprising a single-stranded nucleic acid molecule having a 3′ end, a 5′ end, and a targeting region positioned between the 3′ end and the 5′ end, wherein the targeting region has a sequence targeted to a target region in the gene, and wherein the 3′ end and the 5′ end each have a sequence that enables a formation of a hairpin structure.

76. The method of claim 75, wherein the targeting region of the single-stranded nucleic acid molecule is between about 8 and about 50 nucleotides in length.

77. The method of claim 75, wherein the targeting region of the single-stranded nucleic acid molecule is between about 12 and about 20 nucleotides in length.

78. The method of claim 75, wherein the targeting region of the single-stranded nucleic acid molecule is about 14 nucleotides in length.

79. The method of claim 75, wherein the targeting region of the single-stranded nucleic acid molecule is about 18 nucleotides in length.

80. The method of claim 75, wherein the targeting region of the single-stranded nucleic acid molecule is substantially identical to a target region in the gene.

81. The method of claim 75, wherein the targeting region of the single-stranded nucleic acid molecule is substantially complementary to a target region in the gene.

82. The method of claim 75, wherein the single-stranded nucleic acid molecule is a RNA molecule.

83. The method of claim 75, wherein the single-stranded nucleic acid molecule is a DNA molecule.

84. The method of claim 75, wherein the single-stranded nucleic acid molecule includes nucleotide analogs.

85. The method of claim 84, wherein the nucleotide analogs are selected from the group consisting of phosphorothioates, 2′O-methyl analogs, 2′O-amino analogs, and 2′O-fluoro analogs.

86. The method of claim 75, wherein the single-stranded nucleic acid molecule further comprises a linker sequence positioned between the targeting region and the 5′ end.

87. The method of claim 75, wherein the single-stranded nucleic acid molecule further comprises a linker sequence positioned between the targeting region and the 3′ end.

88. The method of claim 75, wherein the single-stranded nucleic acid molecule further comprises linker sequences positioned between the targeting region and both the 5′ end and the 3′ end.

89. The method of claim 88, wherein the linker sequence includes between about 1 and about 10 nucleotides.

90. The method of claim 89, wherein the linker sequence includes between about 4 and about 8 nucleotides.

91. The method of claim 90, wherein the linker sequence includes about 6 nucleotides.

92. The method of claim 75, wherein the sequence that enables the formation of a hairpin structure includes inverted repeats.

93. The method of claim 75, wherein the sequence that enables the formation of a hairpin structure comprises a loop region including between about 1 and about 10 nucleotides.

94. The method of claim 75, wherein the sequence that enables the formation of a hairpin structure comprises a loop region including between about 2 and about 8 nucleotides.

95. The method of claim 75, wherein the compound comprises a plurality of single-stranded nucleic acid molecules having targeting regions targeted to target regions in a plurality of genes.

96. The method of claim 75, wherein the compound comprises a plurality of single-stranded nucleic acid molecules having targeting regions targeted to a plurality of target regions in a single gene.

97. A compound for silencing a gene comprising:
a single-stranded nucleic acid molecule having the structure:
A¹-L¹-T-L²-A²
wherein:
A¹ comprises a sequence to form a hairpin structure having less than 30 nucleotides;
L¹ comprises a linker sequence having less than 11 nucleotides;
T comprises a targeting sequence targeted to a target region in the gene;
L² comprises a linker sequence having less than 11 nucleotides; and
A² comprises a sequence to form a hairpin structure having less than 30 nucleotides.

98. The compound for silencing a gene of claim 97, wherein:
A¹ comprises a sequence to form a hairpin structure having from about 4 to about 24 nucleotides;
L¹ comprises a linker sequence having from about 4 to about 8 nucleotides;
T comprises a targeting sequence targeted to a target region in the gene having from about 8 to about 50 nucleotides;
L² comprises a linker sequence having from about 4 to about 8 nucleotides; and
A² comprises a sequence to form a hairpin structure having from about 4 to about 24 nucleotides.

99. The compound for silencing a gene of claim 97, wherein:
A¹ comprises a sequence to form a hairpin structure having from about 8 to about 20 nucleotides;
L¹ comprises a linker sequence having less than about 7 nucleotides;
T comprises a targeting sequence targeted to a target region in the gene having from about 12 to about 20 nucleotides;
L² comprises a linker sequence having less than about 7 nucleotides; and
A² comprises a sequence to form a hairpin structure having from about 8 to about 20 nucleotides.

100. The compound for silencing a gene of claim 97, wherein:
A¹ comprises a sequence to form a hairpin structure having from about 10 to about 16 nucleotides;
L¹ comprises a linker sequence having about 6 nucleotides;
T comprises a targeting sequence targeted to a target region in the gene having about 18 nucleotides;
L² comprises a linker sequence having about 6 nucleotides; and
A² comprises a sequence to form a hairpin structure having from about 10 to about 16 nucleotides.

101. The compound for silencing a gene of claim 97, wherein:
A¹ comprises a sequence to form a hairpin structure having from about 10 to about 16 nucleotides;
L¹ comprises a linker sequence having about 6 nucleotides;
T comprises a targeting sequence targeted to a target region in the gene having about
14 nucleotides;
L² comprises a linker sequence having about 6 nucleotides; and
A² comprises a sequence to form a hairpin structure having from about 10 to about 16 nucleotides.

102. The compound for silencing a gene of claim 97, wherein the single-stranded nucleic acid molecule is comprised substantially of ribonucleotides.

103. The compound for silencing a gene of claim 97, wherein the single-stranded nucleic acid molecule is comprised substantially of deoxyribonucleotides.

104. The compound for silencing a gene of claim 97, wherein the single-stranded nucleic acid molecule includes nucleotide analogs.

105. The compound for silencing a gene of claim 104, wherein the nucleotide analogs are selected from the group consisting of phosphorothioates, 2′O-methyl analogs, 2′O-amino analogs, and 2′O-fluoro analogs.

106. The compound for silencing a gene of claim 97, wherein the single-stranded nucleic acid molecule comprises mixed-backbone linkages.

107. The compound for silencing a gene of claim 97, wherein the single-stranded nucleic acid molecule is from about 14 to about 114 nucleotides in length.

108. The compound for silencing a gene of claim 107, wherein the single-stranded nucleic acid molecule is from about 14 to about 72 nucleotides in length.

109. The compound for silencing a gene of claim 108, wherein the single-stranded nucleic acid molecule is about 54 nucleotides in length.

110. The compound for silencing a gene of claim 108, wherein the single-stranded nucleic acid molecule is about 50 nucleotides in length.

111. The compound for silencing a gene of claim 107, wherein the sequence comprising A² terminates in puromycin.

112. The compound for silencing a gene of claim 97, wherein the sequence comprising A² terminates in puromycin.

113. The compound for silencing a gene of claim 97, wherein the, sequences comprising A¹ and A² are configured to form a hairpin structure having a loop of from about 1 to about 10 nucleotides.

114. The compound for silencing a gene of claim 97, wherein the sequences comprising A¹ and A² are configured to form a hairpin structure having a loop of from about 2 to about 8 nucleotides.

115. The compound for silencing a gene of claim 97, wherein the sequences comprising A¹ and A² are configured to form a hairpin structure having a loop of about 4 nucleotides.

116. The compound for silencing a gene of claim 97, wherein the sequence comprising T overlaps the sequence comprising A¹.

117. The compound for silencing a gene of claim 97, wherein the sequence comprising T overlaps the sequence comprising A².

118. The compound for silencing a gene of claim 97, wherein the sequence comprising T overlaps the sequences comprising A¹ and A².
